(19) United States
(12) Patent Application Publication
(10) Pub. No.: US 2006/0104454 A1
(43) Pub. Date: May 18, 2006

(54) Method for selectively picking up a sound signal

(75) Inventors: Jesus Fernando Guitarte Perez, Munich (DE); Gerhard Hoffmann, Eichenau (DE); Klaus Lukas, Munich (DE)

Correspondence Address: STAAS & HALSEY LLP, SUITE 700, 1201 NEW YORK AVENUE, N.W., WASHINGTON, DC, 20005, US

(73) Assignee: SIEMENS AKTIENGESELLSCHAFT, Munich (DE)

(21) Appl. No.: 11/280,226

(22) Filed: Nov. 17, 2005

(30) Foreign Application Priority Data
Nov. 17, 2004 (DE) 102004000043.3

(51) Int. Cl. H04N 7/14; A61F 11/06

(52) U.S. Cl. 381/71.6; 348/14.08

(57) ABSTRACT

A system for selectively picking up a speech signal focuses on a speaker within a group of speakers who wishes to communicate something to the system using an image analysis algorithm to identify, based on a recognition feature, a position of at least one person who wishes to give the system voice commands. The detected position is then used to adapt a directional microphone to the at least one person.

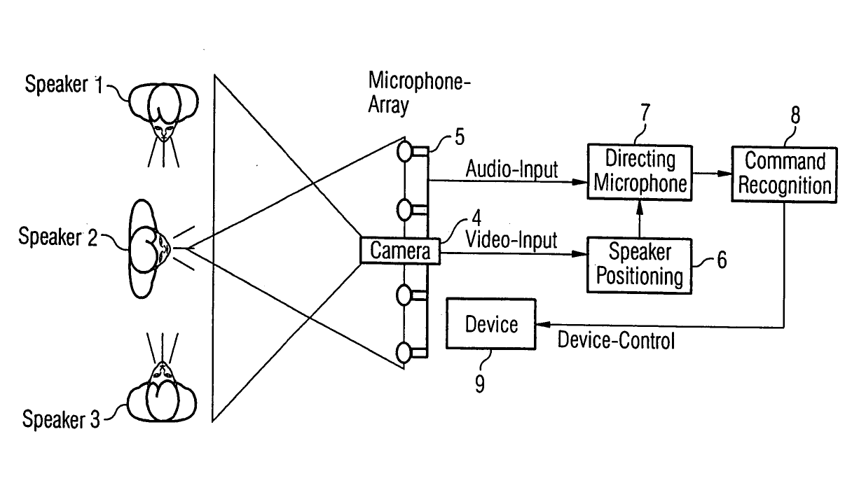

### CROSS REFERENCE TO RELATED APPLICATIONS

This application is based on and hereby claims priority to German Application No. 102004000043.3 filed on Nov. 17, 2004, the contents of which are hereby incorporated by reference.

### BACKGROUND OF THE INVENTION

1. Field of the Invention

The present invention relates to a method, a device and a control program for selectively picking up a sound signal.

2. Description of the Related Art

Voice recognition systems often deliver low recognition rates in a noisy environment. With adjacent or background noise from other speakers in particular it is difficult for the voice recognition system to focus on the main speaker. This is made even more difficult if the environment and situation dictate that close-up microphones, such as headsets, cannot be used. Examples can be found in the automotive area as well as in medical and in industrial environments, where headsets cannot or may not be used.

The use of directional microphones, such as microphone arrays for example, promises a marked improvement in the recognition rates, specifically in environments with a number of speakers and noise sources, since adjacent and/or background noises can be filtered out. For precise focusing of the directional microphone however knowledge of the precise positioning of the speaker is required. This is available in vehicle environments for example, but in other environments on the other hand, such as in the medical environment, the members of a team performing an operation are working in different positions and are also changing their positions during the operation. In the industrial environment too detecting the exact positioning of the person giving the commands is difficult during the operation and installation of systems.

With microphone arrays the different delay times of the audio data picked up with the individual microphones can be used to determine information about the position and the strength of the sound sources. The position of a speaker can thus be determined but no information can be taken from the audio data about the identity of the current speaker to be focused on whose command words are to be executed.

A further approach for determining the position of the speaker is described in F. Asano, Y. Motomura, H. Asoh, T. Yoshimura, N. Ichimura, K. Yamamoto, N. Kitawaki and S. Nakamura, “Detection and Separation of Speech Segment Using Audio and Video information Fusion” in EUROSPEECH 2003, Geneva. This uses visual signals to detect the position of speakers and to align directional microphones to the speaker using the specific position determined. No distinction is made with this method as to which of the speakers wishes to communicate commands to the system.

The disadvantage of the method presented is thus that there is no distinction made as to which of a number of operators who are speaking is giving commands to the system and which operators are merely communicating with other operators. If the commands for speech recognition are thus for example to be issued by different, specific people in a group of operators, it is not possible to use the method previously presented to identify these people.

### SUMMARY OF THE INVENTION

An object of the present invention is thus to specify a method for selectively picking up a sound signal which makes it possible to focus on those people within a group of people whose signals are to be picked up by the system.

According to the present invention, in selectively picking up a sound signal, first, images of persons located at least partly within the range of a directional microphone are picked up by a recording medium. Second, an image analysis algorithm detects at least one position of a person with the aid of a predeterminable recognition feature. Finally, the directional microphone is adapted with the aid of the detected position to the at least one person. Advantageously, with the proposed method the focusing of directional microphones is optimized with the aid of visual information. Thus improvements in the recognition performance are to be expected particularly for environments badly affected by ambient noise through the explicit use of noise filtering. Specifically in medical or industrial environments, where headsets cannot or may not be used, the method can enable new applications for speech recognition to be produced, in which, because of the noise environment, known speech recognition could not previously be used or could only be used to a restricted extent.

Image analysis methods are for example, without restricting the generality of this term, methods for pattern recognition or for detection of objects in an image. Usually with these methods a segmentation is performed in a first step, in which pixels are assigned to an object. In a second step morphological methods are used to identify the shape and/or form of the objects. Finally, in a third step, specific classes are assigned for classification of the identified objects. Typical examples of such methods include handwriting recognition, but also face localization methods.

In accordance with an especially advantageous embodiment of the present invention the image analysis algorithm is embodied as a face localization method. As a recognition feature for identifying that person from a group of people who wishes to issue voice commands to the system, the person turns to face the recording medium. Advantageously in this case a simple recognition feature can be used to indicate the person who wishes to give instructions to the system.

In accordance with a further advantageous development of the present invention, the face of the person is at least partly hidden by a covering means, especially a face mask or a mouth protector. The fact that the person is turning towards the system is detected by the image analysis algorithm with the aid of detection of the edges of the covering means. It is thus also possible to detect that a person is turning towards the system if the person's face can only partly be recognized because of external circumstances and a face localization algorithm can therefore not be used without restrictions. This is for example the case in an operating theater where surgeons may only operate with masks covering their mouth. In the industrial environment too however personnel are often obliged to wear protective clothing.

In accordance with a further advantageous embodiment variants of the present invention the directional microphone can be embodied as a microphone array.

In addition the directional microphone can be adapted to a person with the aid of a beam forming algorithm.

A microphone array usually consists of an arrangement of at least two microphones and is used for directed pick-up of sound signals. The sound signals are recorded simultaneously by the microphones and subsequently shifted in time by a beam forming algorithm in relation to each other such that there is compensation for the delay time of the sound between each individual microphone and the source object to be observed. Addition of the delay time of corrected signals constructively amplifies the components emitted by the source object to be observed whereas the components of other source objects are statistically averaged out.

In accordance with the present invention a device for selectively picking up a sound signal features a recording medium for picking up a person located at least partly within the range of a directional microphone, with an image analysis algorithm detecting at least one position of a person with the aid of a predeterminable recognition feature. The device also features a directional microphone for adapting to the detected position of the person, with a relative position of the directional microphone being known to the recording medium.

In accordance with an advantageous development of the present invention the directional microphone is positioned close to the recording medium. This has the advantageous effect of making it easy to adapt the directional microphone since the person is speaking in the direction of the microphone.

When the inventive control program is executed, first, the program scheduling device causes images of a person located at least partly within the range of a directional microphone to be recorded by a recording medium. Second, an image analysis algorithm detects at least one position of a person with the aid of a specifiable recognition feature. Finally, the directional microphone is adapted with the aid of the detected position to the at least one person.

### BRIEF DESCRIPTION OF THE DRAWINGS

These and other objects and advantages of the present invention will become more apparent and more readily appreciated from the following description of an exemplary embodiment, taken in conjunction with the accompanying drawing of which:

The Figure shows a schematic diagram of a method for video-based focusing of microphone arrays.

### DETAILED DESCRIPTION OF THE PREFERRED EMBODIMENT

Reference will now be made in detail to an exemplary embodiment of the present invention, illustrated in the accompanying drawing.

The Figure shows three loudspeakers 1, 2 and 3, a video camera 4 and a microphone array 5. Boxes 6 to 9 schematically depict the execution sequence for selectively picking up a sound signal.

In the exemplary embodiment illustrated in the Figure, the three speakers 1, 2, 3 who are standing within the range of the microphone array 5 are speaking at the same time. All three speakers 1, 2 and 3 are in this case recorded by the video camera 4 which in this example is embodied as a CCD (Charge Coupled Device) camera. The recorded image is therefore an image recorded by an electronic camera 4 (CCD camera) which can be processed electronically, in which case the recorded image is made up of individual pixels with an assigned gray scale value in each case. Speakers 1 and 3 are in this case not looking into the video camera 4, whereas speaker 2 is turning towards the video camera 4 and is looking to the front into the video camera. In accordance with a preferred embodiment variant of the present invention turning towards the recording medium is a predeterminable recognition feature for the system with which it is notified that speaker 2 would like to give the system a command.

In a speaker positioning operation 6, an image analysis algorithm detects that only speaker 2 is turning towards the video camera and thus wishes to give the system voice commands. In this exemplary embodiment the image analysis algorithm uses a face localization method to detect that only the face of speaker 2 is turned to the front towards the video camera 2.

A geometrical method for analyzing an image to determine the presence and the position of a face initially includes determining segments in the recorded image which exhibit brightness-specific features. The brightness-specific features can for example be bright-dark transitions or dark-bright transitions. Subsequently a relationship between the positions of the segments determined is checked, with a presence of a (human) face, especially at a specific position in the recorded image, being derived if a selection of segments determined exhibits a specific positional relationship. This means that, using the method just described, by analyzing specific areas of the recorded image, namely the segments with brightness-specific features, or to put it more precisely by checking the positional relationship of the segments determined, the presence of a face, especially a human face can be concluded.

In particular segments in the recorded image are determined in which the brightness-specific features exhibit sharp or abrupt brightness transitions, for example from dark to light or from light to dark. These types of (sharp) brightness transitions

can be found for example in a human face, especially where the forehead meets the eyebrows or (for people with light-colored hair) at the transition between the forehead and the shadows of the eye sockets. These types of (sharp) brightness transitions can however also be found at the transition between the upper lip region or lip area for mouth opening or between the mouth opening and the lip area of the lower lip or the lower lip area. A further brightness transition is produced between the lower lip and the chin area, or to put it more precisely as a shadow area (depending on the lighting situation or light incidence) based on a slight protrusion of the lower lip. Preprocessing of the image using a gradient filter enables (sharp) brightness transitions, such as at the eyebrows at the eyes or at the mouth to be especially accentuated and made visible.

To check the positional relationship between the segments determined, in a first investigation for example each of the segments determined is thoroughly investigated as to whether, for a segment to be investigated, a second segment determined exists which essentially lies on a line running horizontally or a line which is essentially running horizontally to the segment determined which has just been investigated. Using a recorded image, consisting of a plurality of pixels, as a starting point, the second segment does not absolutely have to lie on a horizontal line of pixels enclosed by the segment to be investigated, it can also lie higher or lower by a predeterminable amount or pixels in relation to the horizontal line. If a second determined horizontal segment is found, a search is made for a third determined segment which is located below the investigated segment and the second determined segment and for which it is true to say that a distance between the investigated segment and the second determined segment and a distance of a connecting path between the investigated and the second determined segment to the third determined segment exhibits a first prespecifed relationship. In particular a perpendicular to the connection path between the investigated segment and the second defined segment can be defined, with the distance from the third segment (along the perpendicular) to the connection path between the investigated and the second defined segment being included in the first prespecifed relationship. The first investigation just described thus enables the presence of a face to be established, in that the positional relationship between three defined segments is determined. In this case the basic assumption is made that the investigated segment and the second segment determined represents a relevant section of an eyebrows in the face of a person who normally exhibits a clear or sharp light-dark brightness transition from top to bottom and is thereby easily recognizable. The third segment defined represents a segment of a part of the mouth or the boundary area forming shadows between the upper lip and the lower lip. As well as the option of using eyebrows as a clearly-defined segment with brightness-specific features, it is also possible, instead of the eyebrows, to use shadow-forming areas of the eye sockets or the eyes or the iris itself. The method can be expanded as required to additional segments to be investigated, which for example includes detection of pair of glasses or additional verifying features (nose, opened part of the mouth).

In particular the method can also be expanded to members of a team performing an operation who are obliged for hygiene reasons to wear a protective mask over their mouth. In this case, identification of the edges of the mouth protection can be used as an additional recognition feature alongside detection of the eyebrows and/or alternatively an additional optical feature such as for example a horizontal line which simulates a part of the mouth on which the mouth protection is pressed and is included for identification.

Through for example at least two cameras arranged at separate locations (for example CCD line cameras), which simultaneously record images of the person located in the range of the directional microphone and of which the relative position to each other is known, the spatial-position of speaker 2 can be reconstructed.

In a directing microphone operation 7, the microphone array 5 is aligned with the aid of the detected position to speaker 2. This allows the voice signal of speaker 2 to be recorded by the microphone array 5.

A microphone array usually consists of an arrangement of at least two microphones and is used for directional pick-up of sound signals. The sound signals are recorded simultaneously by the microphone array and subsequently shifted by a beam forming algorithm in relation to each other in time such that there is compensation for the delay time of the sound between each individual microphone and the source object to be observed. Addition of the delay time of corrected signals constructively amplifies the components emitted by the source object to be observed whereas the components of other source objects are statistically averaged out.

In a command recognition operation 8, the sound signal recorded by the directed microphone array 5 is evaluated by a speech recognition system. The commands from speaker 2 thus obtained and evaluated are now forwarded to device 9 and executed by the latter.

The concentric arrangement of video camera 4 and microphone array 5 in this example considerably simplifies the orientation of the microphone array 5 to the recognized speaker 2 since the speaker 2 is turning towards the video camera 4 and thereby at the same time to the microphone array 5 to notify the system that he wishes to transmit voice commands to the system.

The invention has been described in detail with particular reference to an exemplary embodiment and examples, but it will be understood that variations and modifications can be effected within the spirit and scope of the invention covered by the claims which may include the phrase “at least one of A, B and C” as an alternative expression that means one or more of A, B and C may be used, contrary to the holding in Superguide v. DIRECTV, 69 USPQ2d 1865 (Fed. Cir. 2004).

What is claimed is:

1. A method for selectively picking up a sound signal, comprising:
recording images of people, located at least partly within range of a directional microphone, on a recording medium;
detecting a position of at least one person based on a predetermined recognition feature using an image analysis algorithm; and
adapting the directional microphone based on the position of the at least one person.

2. A method in accordance with claim 1, wherein the image analysis algorithm is a face localization method.

3. A method in accordance with claim 2, wherein a person at least partially facing towards the recording medium is used as the recognition feature.

4. A method in accordance with claim 3, wherein when the person has a face at least partly covered by a covering formed by at least one of a face mask and a mouth protector, a direction the person is facing is recognized by the image analysis algorithm based on detection of edges of the covering.

5. A method in accordance with claim 3, wherein when the person has a face at least partly covered by a covering formed by at least one of a face mask and a mouth protector, a direction the person is facing is detected by the image analysis algorithm based on an optical feature on the covering.

6. A method in accordance claim 5, wherein the optical feature is at least one of a color and a texture.

7. A method in accordance with claim 6, wherein the directional microphone is a microphone array.

8. A method in accordance with claim 7, wherein the directional microphone is adapted to the person using a beam forming algorithm.

9. A device for selectively picking up a sound signal, comprising:
a recording mechanism recording pictures of at least one person located at least partly within range of a directional microphone, using an image analysis algorithm to detect a position of the at least one person based on a predeterminable recognition feature; and
a directional microphone adapting to the position of the at least one person, with a position of the directional microphone relative to the recording mechanism being known.

10. A device in accordance with claim 9, wherein the directional microphone is substantially co-located with the recording mechanism.

11. At least one computer readable medium storing instructions to control a processor to perform a method comprising:
recording images of people, located at least partly within range of a directional microphone, on a recording medium;
detecting a position of at least one person based on a predetermined recognition feature using an image analysis algorithm; and
adapting the directional microphone based on the position of the at least one person.
